(19) United States
(12) Patent Application Publication
(10) Pub. No.: US 2009/0214503 A1
(43) Pub. Date: Aug. 27, 2009

(54) NEW BACILLUS SUBTILIS STRAIN ATCC® PTA-8805, BIOPRODUCTS CONTAINING SAID STRAIN AND USE OF THE SAME TO CONTROL THE FUNGUS RHIZOCTONIA SOLANI, AN IMPORTANT PLANT PATHOGEN THAT ATTACKS ECONOMICALLY RELEVANT CROPS

(75) Inventors: Luigi Ciampi Panno, Valdivia (CL); Juan Ricardo Fuentes Perez, Valdivia (CL); Marcia Enriqueta Costa Lobo, Valdivia (CL); Juan Pedro Nissen Mutzenbecher, Valdivia (CL); Renate Paula Schobitz Twele, Valdivia (CL); Mauricio Ivan Schoebitz Cid, Valdivia (CL); Patricia Isabel Aguila Torres, Valdivia (CL); Cristina del Vergara Hinostroza, Valdivia (CL)

Correspondence Address: MERCHANT & GOULD PC, P.O. BOX 2903, MINNEAPOLIS, MN, 55402-0903, US

(73) Assignee: Universidad Austral de Chile, Valdivia (CL)

(21) Appl. No.: 12/357,943

(22) Filed: Jan. 22, 2009

(30) Foreign Application Priority Data
Feb. 27, 2008 (CL) 602-2008

(51) Int. Cl. A01N 63/00; C12N 1/20; A01P 3/00

(52) U.S. Cl. 424/93.462; 435/252.5

(57) ABSTRACT

The present application is directed to a new Bacillus subtilis strain deposited under the access number ATCC® PTA-8805, to bioproduct formulations comprising viable cells of the new strain, which can be encapsulated or in a concentrated liquid form, and to the use of these bioproducts to biologically control and decrease the incidence of Rhizoctonia solani, a plant pathogen that attacks economically important crops. The bioproducts are applied in encapsulated form directly over tubers or spraying a concentrated liquid form diluted in water over a plantation, and both formulations are applied in covered furrows.

### OBJECT OF THE INVENTION

The present patent application is directed to a new Bacillus subtilis strain deposited under the access number ATCC® PTA-8805, to bioproduct formulations comprising viable cells of the new strain, which can be encapsulated or contained in a concentrated liquid form, and to the use of these bioproducts to biologically control and decrease the incidence of Rhizoctonia solani, a plant pathogen that attacks economically important crops. Specifically, this use is directed to biologically control and reduce the incidence of Rhizoctonia solani, which is the causal agent of rhizoctoniasis in plants and black scurf in underground propagation clonal organs and underground roots. The bioproducts are applied directly over tubers in the case of the encapsulated form or by spraying a concentrated liquid form diluted in water over a plantation, both applications being carried out in covered furrows.

### TECHNICAL PROBLEM ADDRESSED

Rhizoctoniasis and black scurf are plant diseases that affect economically important crops, especially potato and other crops such as beet and carrot, and is produced by the basidiomycete fungus Rhizoctonia solani. This fungus is responsible for production losses in a broad range of economically important host crops and affects agricultural activities in useful plants around the world. R. solani is present in the majority of the potato producing regions and its development is favored in moist and cold soils.

In the agricultural world, and particularly in Chile, the genus Rhizoctonia causes severe losses and its presence is relevant in potato, carrot, beet, wheat, etc. The control of this fungus is complex and costly, since the available chemicals for this use must be directly applied to the soil or must impregnate the seeds. In the present, they are used with uncertain results that increase production costs and environmental pollution risks.

Hence, biological control is a viable alternative to fight and reduce the effect of R. solani, and, in Chile, this depends on the development and manufacturing of specific biological formulations for the control of plant and tuber diseases.

### BACKGROUND OF THE INVENTION

There are several patents that describe biological control approaches, particularly the activity of B. subtilis as a biocontrol agent. Among them, the patent applications filed by Bergstrom et al., US 20030082792 and US 20050260293, which describe the composition of a B. subtilis isolate as a fertilizer, insecticide, fungicide and nematicide, a method to protect plants against pathogens and the fungicide action of the supernatant obtained from a B. subtilis strain, respectively. These patents are priority to the Chilean patent application No. CL200000628, which was abandoned. The present patent application is different from the applications mentioned above in that the biocontrol strategy is based on the formulation of bioproducts based on a new B. subtilis strain that has superior fungicide features compared to those described in said patents, and it was designed in such a way as to be applied to specific crops such as potato, carrot and beet to control and decrease the abundance of the fungus R. solani and also the diseases caused by this fungus. Likewise, the U.S. Pat. No. 6,060,051 describes the activity of a B. subtilis strain against plant pathogens, but the new B. subtilis strain described by the present patent application is a superior biocontrol agent, since it has a higher antagonist action against the fungus R. solani and thus is able to inhibit the fungus and also to control the diseases caused by the fungus.

For better understanding, clarity and justification of the present patent application, the current state of the art directed to solve the productive problem is further described in the following paragraphs to justify and support this invention.

Potato (Solanum tuberosum L.) is a crop with high agricultural, commercial and nutritional relevance both in Chile and the world. Its use as food is important due to its carbohydrate content and energetic value. Potato has multiple uses such as direct human consumption, cattle feeding, food industry applications (manufacture of dehydrated mashed potato and French fries), starch industry and also distillation industry (alcohol production).

In a world scale, potato is the fourth crop in order of relevance after wheat, rice and corn. Potato is the crop that expands the most its cultivated area in developing countries.

Chile is considered a producer of very high quality potatoes due to its favorable phytosanitary, climatic and soil conditions. This is especially true in the southern part of the country, which exhibits interesting projections for tuber and botanical seed exportations, as long as quarantine and phytosanitary conditions are maintained. The production of this tuber extends across a broad geographical zone in the country, from La Serena in the north to Chiloe Island in the southern territory of the country. The cultivated area concentrates mainly in the IX^{th}, X^{th} and XIV^{th} districts, with a production that reaches more than 50% of the national amount, mainly oriented to the production of certified seed, fresh consumption and industrial use.

Rhizoctonia solani is the causal agent of “rhizoctoniasis” in plants and “black scurf” in potato tubers. R. solani is a fungus that attacks all crops and is present in the majority of the potato producing regions, its development being favored in moist, cold soils with high organic matter content.

The pathogen causes problems in potato sprouts, stolons and underground stems. It induces low shooting and growth weakening, leave yellowing and rolling, and also formation of aerial tubers in the disease phase called “rhizoctoniasis”. The agent forms black or dark brown sclerotia that can be flat and superficial or large and irregular. These are located over the tuber skin. This disease phase is called “black scurf”. Generally, the skin has no abnormalities under the sclerotia. To differentiate sclerotia from earth clumps, they have to be washed out: earth will be removed, while sclerotia will remain adhered. Cracking, malformations, cavities and necrosis can also be present in the tubers. Cancrum in new shoots, stem strangling, lower plant growth and purple leave pigmentation can also be produced. In some very affected plants, aerial axial tubers and basal leaves chlorosis may develop, together with the presence of white fungus mycelium in the base of the stems. Underground stolons can also show brown lesions.

Diseases caused by numerous R. solani variants can be found all over the world, causing losses in many annual crops, including all vegetables, floral species and many major crops.

With regard to the economical importance of the disease in potato production, R. solani produces stolon necrosis, which results in the production of non-marketable tubers due to alterations in size, tuber number and development of deformed tubers.

There are many studies and evidences related to the action of R. solani. Until now, control has been ineffective and often costly. Currently, several methods are used to face this pathology, such as:

a) Seed certification, (Exclusion control system): In the majority of cases, the fungus is transmitted on the tuber skin as sclerotium. Therefore, certified seeds must be selected by removing infested seeds from the group of seeds to be sold, with a very low tolerance to the presence of skin-transmitted diseases. This substantially increases the cost of certified seeds. The present invention stresses out the feasibility of using tubers with low presence of “black scurf”. The assays performed with the experimental bioproduct to assess this assertion involved a terrain with previous potato culture history and medium-infected tubers were purposely used to ensure the presence and a real incidence of the pathology.

b) Culture rotation or soil exchange, (Eradication control system): It has been established that R. solani increases its presence in the soil as long as the same terrain is repeatedly used for potato production. This limits cultivation and affects small producers that do not have many rotation alternatives.

c) Chemicals, (Protection control system): The products used to preventively control R. solani are few and costly, mainly fungicides. Moreover, their action is erratic and act by contact. Furthermore, the high cost of applying this kind of products is well known. Table 1 shows the commercially available fungicide chemicals that fight R. solani-caused diseases.

TABLE 1
Current commercially available chemicals to combat diseases caused by the
fungus R. solani.

| Fungicide trade | | Active | | | |
|---|---|---|---|---|---|
| mark | Manufacturer | ingredient | Toxicity | Crop | Disease |
| CELEST ® 025 | Syngenta | Fludioxonil | Group | Potato | Black scurf (R. solani) |
| FS | Crop | | IV (*) | | |
| PRIORI ® | Protection | Azoxystrobin | Group | Potato | Stem cancrum |
| | AG., Basel, | | IV (*) | | (R. solani) |
| | Switzerland | | | Beet | Root rotting (R. solani) |
| TECTO ® 500 | and | Thiabendazole | Group | Potato seed | Black scurf (R. solani) |
| SC | subsidiaries. | | III (*) | (whole) | |
| | | | | Whole potato | Black scurf (R. solani) |
| | | | | Lawn grass | Brown spot (R. solani) |
| CELEST ® XL | | Fludioxonil + | Group | Other crops | Rhizoctonia spp. |
| 035 FS | | Mefenoxam | IV (*) | (corn, | |
| | | | | sunflower, | |
| | | | | sorghum, | |
| | | | | wheat, barley, | |
| | | | | oat) | |
| MONCUT ® 40 | NIHON | Flutolanil | Group | Potato | Rhizoctonia spp. |
| SC | NOHYAKU | | IV (*) | | |
| | CO. LTD. | | | | |
| | TOKIO, | | | | |
| | JAPAN. | | | | |
| ROVRAL ® 4 | Bayer | Iprodione | Group | Potato and | Rhizoctonia sp. |
| FLO | CropScience | | IV (*) | others (pepper, | |
| | Brazil. | | | lettuce, melon, | |
| | | | | pumpkin and | |
| | | | | ornamentals) | |
| RUKON 50 | JIANGSU, | Iprodione | Group | Tomato | Rhizoctonia |
| WP ® | CHINA | | IV (*) | | |
| ROVCAP | Bayer | Iprodione + | Group | Potato | Rhizoctonia sp. |
| | CropScience | Captan | IV (*) | | |
| | A.G. and | | | | |
| | subsidiaries. | | | | |
| CERCOBIN ® M | Nippon Soda | Methyl | Group | Soil application | Rhizoctoniasis |
| | Co. Ltd. | thiophanate | IV (*) | | |
| | Japan. | | | | |

(*) Group III. Low risk products./Group IV. Usually risk-free products.

Source: AFIPA, IMPPA and SAG. Manual Fitosanitario 2006-2007 [Phytosanitary Handbook 2006-2007]. Faculty of Agronomy and Forestry Engineering. Pontifical Catholic University of Chile. 1160 p.

The possibility of controlling R. solani using a biological agent is an alternative to the traditional chemical control approach and is simultaneously able to cut costs with an often equal effectiveness.

The use of microorganisms with biological purposes has become an effective alternative to control pathogenic agents in plants through the last years. Many agents have been described up to this date with biocontrolling capability, and the genus Bacillus stands up among them as one of the most efficient producers of antibiotic substances.

Regarding the genus Bacillus, these microorganisms are easily isolated from soil or air. One of their characteristics is their ability to induce the formation of endospores. B. subtilis is exceptional among the genus Bacillus for its potential to produce several antibiotics (Yoshida, S., Hiradate, S., Tsukamoto, T., Hatakcda, K. and Shirata, A. 2001. Antimicrobial activity of culture filtrate of Bacillus amyloliquefaciens RC-2 isolated from mulberry leaves. Phytopathology, 91(2): 181-187). These are produced by specific strains and are not necessary for the microorganism's survival. The synthesis of these peptides often begins at the end of the exponential growth phase, when maximal concentration has been achieved and after cellular development has stopped. Several authors suggest that microorganisms can synthesize antibiotics in their growth phase. It has also been suggested that antibiotic synthesis is probably due to depletion of nutrients necessary for cell development. This nutrient limitation stimulates differentiation, which leads to endospore formation in Bacillus.

Biopesticides are products containing a microorganism as an active ingredient or are extracted from a living organism through procedures that do not alter its chemical composition. A biopesticide can comprise the entire extracted matter or a part of it, concentrated or not, and can be mixed with adjuvant substances or not. These products are an excellent alternative to conventional toxic chemicals used for pest and weed management. Thus, in developed countries the research has focused mainly in the production of biopesticides formulated to compete with existing agrochemicals or to fill a gap where these chemical pesticides are inadequate.

Microbial inocula are interesting in two main agricultural fields: the biological control of plant diseases and the improvement of plant growth through an increase in the availability of nutrients. The use of microorganisms to improve soil quality by improving the physical structure thereof and hence optimizing the growth of the crop, is also being investigated.

One of the main, if not the most important, of the problems regarding the management of microorganisms useful for agriculture and man, is related to the massive transport of bacteria from the lab or cell production facilities to field locations where they are to be incorporated into real and concrete productive processes. This is necessarily related to the problem of ensuring an adequate survival of bacterial inocula during the storage period prior to its use and also to achieving an optimal inoculation at the moment of cultivation and/or crop plantation in the field.

An adequate release system should enable the bioprotecting microorganisms to colonize effectively the rhizosphere and, simultaneously, the root surface of the plant or the surface of any other substrate. However, this is only possible after suitable transportation and a favorable settlement of the microorganisms in the soil. Such an objective requires a bacterial formulation able to duly protect the antagonist cell integrity from the moment they are produced in vitro to their final application in the field, through formulation processes and storage periods. Furthermore, the inoculated bacteria should have an adequate availability of substances required to multiply themselves, in order to develop and proliferate in the rhizosphere up to levels suitable to ensure the desired biological role.

Considering the aforementioned points, the concept involving the adequate transportation of antagonistic bacteria into the field must be broadened by incorporating material elements or vehicles that could really contribute to bioinoculation and by defining a suitable formulation and storage process. Therefore, the use of a highly efficient or genetically improved strain is insufficient by itself to guarantee an effective biological control. Hence, the selection of a system to release antagonistic microorganisms in the field is a determinant factor for the success or failure of the bioinoculation of disease control agents (Harman, G. 1991. Seed treatment for biological control of plant disease. Crop Protection 10:166-171). This is included in the final disclosure to manufacture a product that is readily usable by farmers and does not involve special machines or efforts to be applied in crop fields.

Encapsulation or microencapsulation is a form of cellular immobilization. Capsules are spherical particles with semipermeable membranes that are defined as a special form of packing in which a particular material can be individually covered to protect it from environmental and unfavorable influences. In a broad sense, encapsulation provides a mean to pack, separate and store materials at a microscopic scale, and to release these materials afterward under controlled conditions.

Cellular encapsulation comprises the inclusion of microorganisms within any type of gel matrix. Living cell trapping within matrixes, such as alginate matrixes, consists in producing a cell suspension, mixing the cells with a sodium alginate solution and dropping the mixture into a solution that contains polyvalent cations, usually Ca²⁺. Drops immediately become solid spheres, trapping the cells within a 3D matrix of ionically-linked alginate. Alginate fulfills many of the requirements of a good inoculant: it is dry, readily usable, uniform, biodegradable and non-toxic. It is also able to contain a high bacterial population and to release the microorganisms into the soil slowly over prolonged periods.

The present invention refers to a new Bacillus subtilis strain deposited in ATCC® under the access number ATCC® PTA-8805, and to the formulation and the use of bioproducts comprising native viable cells of the new strain to biologically control and decrease the incidence of Rhizoctonia solani, the causal agent of rhizoctoniasis in plants and black scurf in underground propagation clonal organs and underground roots, both being diseases for which no effective control is currently available. In the present, chemical treatment is used with uncertain results as a control mean, which increase production costs and environmental pollution risks. Furthermore, some biological products containing B. subtilis that are used to control Rhizoctonia spp. are available in the market, but those are not designed for crops such as potato, have only minor antagonistic effects and are different from the bioproducts proposed in this document both in the formulation as in the strain and antagonistic ability.

It is worth to mention that in agriculture the control of pathogens and the diseases caused by them vary considerably according to the crop affected, i.e. pathologies cannot be faced using the same strategies and therefore the products used to control pathogens, these being fungi or bacteria, do not have a universal effect. Due to this reason, the bioproducts described in the present patent application are effective in the biological control of R. solani and the diseases caused by this fungus.

The products proposed herein are biological and industrially scalable. These can be applied in productive situations at the moment of planting a potato field or any other crop field wherein R. solani could be a threat, such as beet and carrot. This is particularly applicable in those soils and locations where this agent is also a production limiting factor.

### DETAILED DESCRIPTION OF THE INVENTION

The present patent application discloses a new Bacillus subtilis strain ATCC® PTA-8805, bioproduct formulations comprising viable cells of the native Bacillus subtilis strain ATCC® PTA-8805, encapsulated or in concentrated liquid forms, and the use of these encapsulated or concentrated liquid bioproducts to biologically control and decrease the incidence of Rhizoctonia solani, the causal agent of rhizoctoniasis in plants and black scurf in underground propagation clonal organs and underground roots.

The native Bacillus subtilis strain ATCC® PTA-8805 was isolated from soil samples of the botanical garden of the city of Valdivia (Chile) and was freeze-dried in vitro as part of the strain collection of the Biomaterials Laboratory of the Faculty of Agricultural Sciences of the Austral University of Chile. The strain exhibited a tested and duly assessed antagonistic action against R. solani and has been identified as a Bacillus subtilis strain. It was deposited in ATCC® on Dec. 11, 2007 under the accession number ATCC® PTA-8805. This bacterial strain constitutes the biological basis of the encapsulated and concentrated liquid bioproducts disclosed in the present patent application.

The B. subtilis strain ATCC® PTA-8805 was characterized at the Austral University of Chile by morphology description, Gram staining and biochemical assays usually used in microbiology and widely known (Table 2). Likewise, the strain was identified by API galleries, a standardized system for identification of bacterial and yeast genera and species by associating a biochemical assay gallery with a database, using the API® Systems 50 CHB/E and 20E.

Colonies of the strain ATCC® PTA-8805 are uprisen, have irregular edges, mucous consistency, opaque appearance and white color. Elongated, Gram-positive, irregularly grouped bacilli approximately 1.5×0.6 μm in size can be observed under clear field microscopy. Additionally, strain ATCC® PTA-8805 sporulates at 4° C. after 48 hours of growth in Peptone Agar (PA). The medium was prepared with distilled water by dissolving 24 g/L of PA medium (Merck), comprising: 5 g/L casein peptone; 3 g/L meat extract; 1 g/L D(+)-glucose; 15 g/l agar, at a pH of 7.0±0.1. The medium was autoclaved at 121° C. for 15 min at 101.325 kPa (1 atm).

Biochemical assays determined that this strain belongs to the Bacillaceae family, genus Bacillus, species subtilis; the assays were comparatively run also on the standard Bacillus subtilis strain ATCC® 9799 to check the correct identification of the strain. Table 2 shows that strain ATCC® PTA-8805 has the typical features of a B. subtilis strain, which match exactly the standard strain ATCC® 9799.

Additionally, the identification system API® 50 CHB/E combined with the system API® 20 E confirmed that the strain ATCC® PTA-8805 shows 82.8% identity similarity with Bacillus subtilis and 16.7% with Bacillus amyloliquefaciens, as shown in Table 3. The Bio Merieux database determined a good identification with the genus Bacillus.

Simultaneously, the antagonistic effect of the strain ATCC® PTA-8805 was confirmed by confronting it with R. solani. The antagonism inhibition halo radius was approximately 12 mm, which was compared in parallel with the result of an antagonism assay with the standard strain ATCC® 9799, which showed a much lower antagonistic effect with an inhibition halo radius of 3.8 mm. Both assays were carried out in Agar Potato Dextrose (APD) medium. This medium is prepared with distilled water by dissolving 39 g/L of APD medium (Merck), comprising: 4 g/L potato infusion (prepared from 200 g potato), 20 g/L D(+)-glucose and 15 g/L agar-agar, with a pH of 5.6±0.1. The medium was autoclaved at 121° C. for 15 min at 101.325 kPa (1 atm).

TABLE 2
Results of the biochemical assays carried out on the strain ATCC ®
PTA-8805 in comparison with the strain ATCC ® 9799
after an incubation of 24 and 72 h.

| | Strain ATCC ® | | Strain ATCC ® | |
|---|---|---|---|---|
| | PTA-8805 | | 9799 | |
| Assay** | 24 h | 72 h | 24 h | 72 h |
| Endospore production | + | + | + | + |
| Indole production | − | − | − | − |
| Growth on citrate | − | + | − | + |
| Starch hydrolysis | +++ | +++ | +++ | +++ |
| Growth at 32° C. | + | + | + | + |
| Growth at 65° C. | − | − | − | − |
| Glucose utilization | + | + | + | + |
| Voges-Proskauer test | − | + | − | + |
| Oxydase test | + | + | + | + |
| Catalase test | +++ | +++ | +++ | +++ |
| Growth in 7% NaCl | + | + | + | + |
| Urea utilization | − | − | − | − |
| Anaerobic growth | − | − | − | − |

| Morphology and Gram | Gram(+) rods or bacilli | |
|---|---|---|
| staining | | |
| Size | 1.5 × 0.6 μm | 2.0 × 0.6 μm |

**Assays carried out according to materials and methods described in Gordon, R. E., Haynes, W. C. & Pang, C. H. 1973. The Genus Bacillus. Agriculture Handbook 427. US Department Agriculture Washington DC. USA.

TABLE 3
Identification percentages of significant taxons for the strain
ATCC ® PTA-8805.

| Significant taxons | Identification percentages |
|---|---|
| B. subtilis | 82.8% |
| B. amyloliquefaciens | 16.7% |

Next, encapsulated and concentrated liquid formulations comprising mainly viable cells of the native B. subtilis strain ATCC® PTA-8805 will be described.

The encapsulated bioproduct is obtained from an ecnapsulation-suitable matrix comprising native viable cells of the strain ATCC® PTA-8805, molasses culture medium and sodium alginate. The molasses culture medium is a liquid medium used to culture B. subtilis, which is characterized by a demonstrated high biomass yield, low cost and easiness of preparation, and also maintains the antagonistic ability of the strain. The medium comprises: water; 20 g/l sugar beet molasses; 2 g/L sucrose; 1 g/L yeast extract; 5 g/L ammonium sulfate ((NH₄)₂SO₄) and 1 ml/L silicone antifoaming agent, further adding 0.38 g/L dipotassium hydrogen phosphate (K₂HPO₄) and 11.25 g/L potassium dihydrogen phosphate (KH₂PO₄) to adjust the pH to 5.5. The prepared medium was autoclaved at 121° C. for 15 minutes at 101.325 kPa (1 atm).

The encapsulation matrix consists of native viable cells of the strain ATCC® PTA-8805, molasses culture medium and 2% sodium alginate, and is used for cell trapping since sodium alginate acts as a coagulant when put into contact with calcium gluconate, which is carried out by dropping this matrix solution into a 0.1 M calcium gluconate solution. Drops immediately become solid spheres, trapping the strain ATCC® PTA-8805 cells within a 3D matrix of ionically-linked alginate. This procedure is carried out in a bioencapsulator device, e.g. a Nisco® Var-D (www.nisco.ch).

The encapsulated bioproduct formulation has a concentration of viable cells of the strain ATCC® PTA-8805, the active ingredient of the product, of 10⁵-10⁸ CFU/g.

The encapsulated bioproduct is used fresh, i.e. as fresh biocapsules, to biologically control and reduce the incidence of Rhizoctonia solani, the causal agent of rhizoctoniasis in plants and black scurf in tubers such as potato and other crops such as beet and carrot. The bioproduct application method consists in applying fresh biocapsules over recently planted seeds on the same plantation day, on tubers and in covered furrows, in an amount around 1.5-2 g/tuber, i.e. 2-3 biocapsules/tuber.

The liquid concentrated bioproduct is obtained from a liquid medium consisting of native viable strain ATCC® PTA-8805 cells and molasses culture medium. This medium is decanted at 24° C. for 48 hours and the supernatant is separated after this period, thus producing the concentrated liquid bioproduct.

The concentrated liquid bioproduct formulation has a concentration of viable cells of the strain ATCC® PTA-8805, the active ingredient of the product, of 10¹⁰-10¹² CFU/mL.

The concentrated liquid bioproduct is used as a liquid suspension to biologically control and reduce the incidence of Rhizoctonia solani, the causal agent of rhizoctoniasis in plants and black scurf in potato tubers and other crops such as beet and carrot. The concentrated liquid bioproduct has a shelf life of approximately 1 year, estimated based on B. subtilis strain ATCC® PTA-8805 cell counts, population effectiveness and antagonistic activity. The bioproduct application method consists in spraying the product diluted in water on the tubers, in covered furrows, every 4 weeks until harvest. Before applying the bioproduct, it must be stirred to obtain a homogeneous suspension and kept at a temperature of 30-32° C. for at least 3 hours, and then the mixture must be conveniently diluted in cold boiled water up to a concentration of 10⁵-10⁶ CFU/mL.

### APPLICATION EXAMPLES

### Example 1

Biological component Bacillus subtilis strain ATCC® PTA-8805: Strain isolation and characterization.

The B. subtilis strain ATCC® PTA-8805 was isolated from soil samples of the botanical garden of the Austral University of Chile at Valdivia. The strain was freeze-dried in vitro as part of the strain collection of the Biomaterials Laboratory of the Faculty of Agricultural Sciences of the Austral University of Chile. It has a tested and demonstrated antagonistic action against R. solani. The strain was deposited in ATCC® on Dec. 11, 2007 under the access number ATCC® PTA-8805.

To carry out the description of the isolated strain, a pure culture was firstly obtained by streaking on PA plates and incubating at 28° C. for 48 hours. Once the pure culture was obtained, a description of the macroscopic colonies was carried out by directly observing the plate.

The microscopical description and morphologic characterization was performed by using Gram staining and clear field and electronic microscopy.

Biochemical assays usually employed and well known in the microbiology field were used (Table 2).

Additionally, the strain ATCC® PTA-8805 was identified using the API® 50 CHB/E galleries combined with the system API® 20 E.

Colonies of the strain ATCC® PTA-8805 in Petri dishes on PA medium were uprisen, with irregular edges, mucous consistency, opaque appearance and white color.

Under clear field microscopy, morphological and staining characteristics of the strain were determined. The ATCC® PTA-8805 microorganisms are elongated Gram-positive bacilli, irregularly grouped, with a size around 1.5×0.6 μm, and formed endospores at 4° C. after 48 hours of growth in PA.

The biochemical assays determined that the strain ATCC® PTA-8805 belongs to the Bacillaceae family, genus Bacillus, species subtilis. The results of these assays are shown in Table 2.

Subsequently, API galleries confirmed that the strain ATCC® PTA-8805 shows 82.8% identity similarity with Bacillus subtilis and 16.7% with Bacillus amyloliquefaciens, as shown in Table 3. The Bio Merieux database determined a good identification with the genus Bacillus.

### Example 2

Comparison of the antagonistic effect of the standard B. subtilis strain ATCC® 9799 and the strain ATCC® PTA-8805 against the fungus R. solani.

The antagonism assay was performed in a plate by confronting the standard B. subtilis strain ATCC® 9799 and the B. subtilis strain ATCC® PTA-8805, the biological base of the bioproducts described in this patent application, against R. solani strains.

The antagonism assay was carried out by laying a PDA disc with R. solani mycelium at the center of a Petri dish with PDA. At the periphery of the plate, two wells with a sterile cavity of around 1 cm in diameter were made and 100 μL of the B. subtilis strain ATCC® PTA-8805 were deposited within and left to dry for 20 min in a laminar flow chamber. Subsequently, the plate was incubated at 25±2° C. for 3 to 5 days. The assay was performed in duplicate and a negative control with R. solani alone was used. Finally, the radii of inhibition halos were measured.

The results are shown in Table 4, where it is evident that the antagonistic activity of the strain ATCC® PTA-8805 against the fungus R. solani is clearly higher, with an inhibition halo of 12.18 mm, approximately three times higher than the radius of the halo produced by the standard B. subtilis strain ATCC® 9799. This demonstrates that the strain ATCC® PTA-8805, the biological base of the bioproducts described in this patent application, has a proved antagonist activity against R. solani, which constitutes an advantage over other B. subtilis strains.

TABLE 4
Results of the antagonism assay of the standard B. subtilis strain
ATCC ® 9799 and the strain ATCC ®
PTA-8805 against the fungus R. solani.

| | Measure of the inhibition halo radius |
|---|---|
| B. subtilis strain | after 72 hours of incubation |
| ATCC ® 9799 | 3.8 mm |
| ATCC ® PTA-8805 | 12.18 mm |

### Example 3

Procedure to Manufacture the Bioproducts.

The procedure to manufacture the encapsulated or concentrated liquid bioproducts follows the steps of:

a) Activating the Antagonistic Strain ATCC® PTA-8805 and Preparing an Inoculum.

The bacteria used for the initial B. subtilis strain ATCC® PTA-8805 culture must have good viability and antagonistic capability against R. solani. When the culture was started, pure freeze-dried cultures of B. subtilis strain ATCC® PTA-8805 cells were available. The freeze-dried strain (0.1 g) was reactivated by inoculation in 1 mL of sterile Protease Peptone Broth (PPB), using a sterile syringe introduced through the cap of the tube that contained the freeze-dried strain, which was disinfected with 70° alcohol, and then the inoculated culture was incubated at 28° C. for 48 hours. PPB (Merck) was prepared in distilled water by dissolving 18 g/L of the solid medium comprising: 10 g/L casein peptone, 3 g/L meat extract and 5 g/L sodium chloride, with a pH of 7.0±0.1. The prepared medium was autoclaved at 121° C. for 15 minutes at 101.325 kPa (1 atm). After 48 hours, the reactivated freeze-dried cells were streaked on PDA and incubated at 32° C. for 24 hours.

Subsequently, the culture purity and morphology were assessed as a quality control for the strain.

Gram staining was performed on a colony of the strain ATCC® PTA-8805 isolated from the plate to check the purity and morphology. Gram-positive bacilli with endospores were observed.

Simultaneously, the antagonistic effect of the strain ATCC® PTA-8805 was confirmed by confronting it with R. solani. The antagonism test was carried out according to the procedure described previously in the Example 2. The antagonism inhibition halo radius caused by the B. subtilis strain ATCC® PTA-8805 against R. solani in PDA medium was around 12 mm in average.

An inoculum was prepared for massive cell multiplication from the pure B. subtilis strain ATCC® PTA-8805 reactivated in PPB. To this purpose, 10% was inoculated in molasses culture medium. The medium comprises: water; 20 g/l sugar beet molasses; 2 g/L sucrose; 1 g/L yeast extract; 5 g/L ammonium sulfate ((NH₄)₂SO₄) and 1 ml/L silicone antifoaming agent; additionally, 0.38 g/L dipotassium hydrogen phosphate (K₂HPO₄) and 11.25 g/L potassium dihydrogen phosphate (KH₂PO₄) were added to adjust the pH to 5.5. It was autoclaved at 121° C. for 15 minutes at 101.325 kPa (1 atm) and was incubated in an orbital shaker at 120 rpm and 32° C. for 48 hours or until reaching an optical density reading (DO_{600 nm}) of 1 (equivalent to approximately 10⁸ CFU/mL). In this way, 600 mL of inoculum was obtained for growth in liquid medium. A 5 mL sample was taken to count the viable cells (CFU/mL), assess the optical density of the culture (DO_{600 nm}) and prepare a new inoculum.

b) Massively Multiplying the Strain ATCC® PTA-8805.

The antagonist strain ATCC® PTA-8805 was cultured in molasses growth medium in an orbital shaker with continuous rotation at 220 rpm and at 32° C., firstly in 1,000 mL Erlenmeyer flasks with 200 mL of culture medium. In each case, a primary culture was prepared from one isolated bacterial colony in 40 mL flasks; these cultures were incubated for 48 hours (or until reaching a DO of 1 at 600 nm). Subsequently, the primary culture was inoculated into the culture medium in a final volume of 400 mL. The estimation of bacterial growth was performed by measuring the optical densities of the cultures at a wavelength of 600 nm with a spectrophotometer.

Large-scale production of the B. subtilis strain ATCC® PTA-8805 was carried out in a 14 L fermenter (Microferm, New Brunswick Sci., USA) in a batch culture with minimal requirements for growth of B. subtilis, i.e. aeration system and temperature and pH control. The culture growth was monitored by taking samples at 0, 24 and 48 hours and determining culture purity (Gram staining), count of colony forming units (CFU/mL) and optical density (DO_{600 nm}) for each sample.

Large scale production was carried out by using 10 L of molasses culture medium and 1.1 L of inoculum, at a temperature of 30° C., with an airflow of 11 L/min, 1 vvm, stirring at 250 rpm, pH 5.5 regulated with 5 N NaOH and 5 N HCl, 10 mL of silicone antifoaming agent and for 48 hours, with no matter exchange with the surrounding environment except gases.

c) Preparing the Formulation to Obtain the Bioproduct

The process to prepare the formulation depend on the desired bioproduct, either encapsulated or concentrated liquid.

1.-Process to Manufacture the Encapsulated Bioproduct:

a) Centrifuging the Culture to Obtain a Cell Pellet.

To this end, a volume of 600 mL of the culture obtained in the massive cell multiplication, with a cell concentration of 10⁸ CFU/g was centrifuged at 10,000 rpm for 15 minutes at room temperature. The supernatant was removed and the cell pellet was extracted.

b) Preparing the Encapsulation Matrix.

The encapsulation matrix comprised molasses culture medium, 2% sodium alginate and the cell pellet of the strain ATCC® PTA-8805 obtained in step (a). The first step was the suspension in 600 mL of molasses culture medium and 2% sodium alginate (12 mL) for 12 hours with a magnetic stirrer (VELP® SCIENTIFICA) at around 20° C.

c) Manufacturing the Encapsulated Bioproduct:

To manufacture the encapsulated bioproduct, sodium alginate was reacted with 0.1 M calcium gluconate present in the container that received the mixture in a Nisco® Var-D bioencapsulator (www.nisco.ch), thus causing the matrix to coagulate when this two components met each other. This transformed each matrix drop in a biocapsule with a constant diameter of around 3 mm. The capsules were stirred for 15 minutes in the solution to achieve the desired gel firmness and the biocapsules were removed with a sterile strainer once coagulated. Then, the biocapsules were washed with 600 mL of distilled sterile water to remove the excess of calcium gluconate, were collected in sterile flasks and kept in distilled water.

The following parameters were used for encapsulation in the Nisco® Var-D bioencapsulator (www.nisco.ch): peristaltic pump rate: 15 rpm; nozzle diameter: 400 μm; frequency: 0.90 KHz; amplitude: 100%; and stroboscopic light: 100%. Using these encapsulation parameters, size-homogeneous, spherical biocapsules were obtained with no collapse in the encapsulation system.

2) Process to Manufacture the Concentrated Liquid Bioproduct:

To manufacture the concentrated liquid bioproduct, the previously mentioned steps to activate and massively multiply the strain were carried out.

Once achieved the maximum growth of the batch culture in the massive multiplication step, the culture of the strain ATCC® PTA-8805 was concentrated by decantation at 24° C. for 48 hours, to generate viable active bacterial biomass. After decanting, the supernatant was removed. The concentrated bioproduct had a cell count of 10¹² CFU/mL and showed a very marked inhibition halo radius (see Example 2, Table 4). The concentrated liquid bioproduct was maintained at room temperature (20° C.), unshaken and sealed in 500 mL bottles. This bioproduct has a shelf life of approximately 1 year, estimated based on B. subtilis strain ATCC® PTA-8805 cell counts, population effectiveness and antagonistic activity.

### Example 4

Comparison of the antagonistic effect of the B. subtilis strain present in biological products of the present invention with commercially available bioproducts against the fungus R. solani.

The antagonism assay was performed in a plate by confronting the B. subtilis strain ATCC® FZB24, base of the biological product Rhizo Plus®, the B. subtilis strain ATCC® QST 713, base of the biological product Serenade®, and the B. subtilis strain ATCC® PTA-8805, the biological base of the bioproducts described in this patent application, against R. solani strains.

The procedure followed to carry out the antagonism assay was described previously in the Example 2.

The antagonism assay was evaluated by measuring the inhibition halo radius produced at 72 hours of incubation. The results are shown in Table 5.

TABLE 5
Results of the antagonism assays of B. subtilis strains present
in biological products against the fungus R. solani.

| | Measure of the inhibition halo radius |
|---|---|
| B. subtilis strain | after 72 hours of incubation |
| FZB24 | 6.84 mm |
| QST 713 | 6.22 mm |
| ATCC ® PTA-8805 | 12.21 mm |

In Table 5 it is evident that the antagonistic activity of the strain ATCC® PTA-8805 against the fungus R. solani is higher, with an inhibition halo of 12.21 mm, approximately two times higher than the radius of the halos produced by the strains FZB24 and QST 713. The strain ATCC® PTA-8805 is the biological base of the bioproducts disclosed in this patent application, thus proving the superior antagonistic ability of the strain ATCC® PTA-8805 against R. solani and the diseases caused by this fungus.

### Example 5

Application of the Encapsulated Bioproduct in the Field.

The application of the encapsulated bioproduct in the field was carried out the same day when potatoes were sown. Fresh encapsulated formulation was used, i.e. fresh biocapsules with a final concentration of 10⁵ CFU/g were applied over recently planted seeds in covered furrows, in an amount around 1.5-2 g/tuber, i.e. 2-3 biocapsules/tuber.

Evidence in Chile of Field Assays to Test the Efficacy of the Encapsulated Bioproduct.

The field assays to test the function of the biological treatments with the encapsulated bioproduct were carried out on November-December 2006 and January-February-March 2007 in an evaluation carried out at the Santa Rosa Experimental Station of the Austral University of Chile. To this aim, the following field situations were used:

a) A Field with a History of Repeated Potato Culture.

b) Potato Seed with Medium R. Solani Infestation (5 to 10%).

The assay was performed under the former conditions to ensure the presence and real incidence of the pathology, in order to test the results of the biological control approach disclosed in this patent application.

The results obtained in the application assays, shown in Table 6, considered percentage of healthy tubers, weight and number of commercial quality tubers per plant, and demonstrate that better results were obtained with the application of the encapsulated bioproduct in covered furrows, including:
Higher amount of healthy tubers.
Higher tuber weight per plant.
Higher tuber number per plant.

TABLE 6
Percentage, weight and number of tubers harvested per treatment.

| | Healthy tubers | Tuber weight | Number of tubers |
|---|---|---|---|
| Treatment (*) | (%) | (g/plant) | per plant |
| Encapsulated, covered | 58.12* | 678.28* | 6.00* |
| furrow | | | |
| Untreated control | 24.00 | 296.78 | 2.70 |

Wherein:
*= is statistically different from the control according to Tukey's test.
(*) = Encapsulated: alginate matrix with trapped bacteria of the strain ATCC ® PTA-8805, in a cellular concentration of 10⁵ CFU/g.

When comparing the results of the application of capsules in covered furrows with the untreated control, it is evident that in the control situation there were less healthy tubers, lower weights and lower number of tubers per plant. The characteristics shown by the untreated control are typical effects of the fungus R. solani. Therefore, this evidence demonstrates that this agent can be controlled effectively by using the approach disclosed in the present patent application.

### Example 6

Application of Concentrated Liquid Bioproduct.

The concentrated liquid bioproduct was stirred until complete homogenization and kept at approximately 32° C. for 3 hours before application. Subsequently, 1 mL of the concentrated liquid bioproduct was diluted in 1 L of cold boiled water at a concentration of 10⁵ CFU/mL and was applied in the field by spraying over potatoes in covered furrows every 4 weeks during the development of the potato crop and until harvest.

Evidences in Chile of field assays to test the efficacy of the concentrated liquid bioproduct.

The field assay was performed on November-December 2006 and January-February-March 2007, at the Santa Rosa Experimental Station of the Austral University of Chile in Valdivia, and considered the percentage of commercial-quality healthy tubers, weight and number of commercial-quality tubers per plant.

The assay was carried out in a field that previously supported potato plantations. Furthermore, potato seeds with medium black scurf infestation (5-10%) was used in order to ensure the effective presence and incidence of the disease.

The obtained results can be appreciated in Table 7, demonstrating that a liquid application every 4 weeks yields the best results with a statistically significant higher number of healthy tubers per plant and higher tuber weight per plant. The effect of R. solani is appreciated in the untreated control, which had a lower percentage of healthy tubers per plant and a lower tuber weight per plant. Therefore, this demonstrates that the biocontrol approach disclosed in this patent application is able to attack effectively the target pathogen.

TABLE 7
Percentage, weight and number of tubers harvested per treatment.

| | Healthy | Tuber weight | Number of tubers per |
|---|---|---|---|
| Treatment (*) | tubers (%) | (g/plant) | plant |
| Liquid every 4 weeks | 73.57* | 702.75* | 4.50* |
| Untreated control | 47.91 | 264.20 | 4.55 |

Wherein:
*= is statistically different from the control according to Tukey's test.
(*) Liquid = suspension of cells of the strain ATCC ® PTA-8805in molasses culture medium at a cell concentration of 10⁵ CFU/mL.

1. An isolated bacterial strain belonging to genus Bacillus and species subtilis, deposited on Dec. 11, 2007 under the accession number ATCC® PTA-8805, wherein said strain are Gram-positive, endospore-forming, bacillary bacteria, and they show proven antagonist activity against Rhizoctonia solani, a plant pathogen that affects economically important crops.

2. An encapsulated bioproduct formulation that comprises viable cells of the strain ATCC® PTA-8805, molasses culture medium and sodium alginate.

3. An encapsulated bioproduct formulation according to claim 2, wherein said bioproduct formulation comprises cells of the strain ATCC PTA-8805 at a concentration ranging from 10⁵ to 10⁸ CFU/g, molasses culture medium and 2% sodium alginate.

4. An encapsulated bioproduct formulation according to claim 3, wherein the molasses culture medium comprises: water, 20 g/L sugar beet molasses, 2 g/L sucrose, 1 g/L yeast extract, 5 g/L ammonium sulfate ((NH4)2SO4), 1 ml/L silicone antifoaming agent, 0.38 g/L dipotassium hydrogen phosphate (K₂HPO₄) and 11.25 g/L potassium dihydrogen phosphate (KH₂PO₄).

5. The method of using the bioproduct formulation according to claim 2, wherein said bioproduct formulation is used to biologically control and decrease the incidence of Rhizoctonia solani, the causal agent of rhizoctoniasis in plants and black scurf in underground propagation clonal organs and underground roots.

6. The method of using the bioproduct formulation according to claim 5, wherein said underground propagation clonal organs are potato tubers.

7. The method of using the bioproduct formulation according to claim 5, wherein said underground roots are carrots or beets.

8. A concentrated liquid bioproduct formulation that comprises viable cells of the strain ATCC® PTA-8805 and molasses culture medium.

9. A concentrated liquid bioproduct formulation according to claim 8, wherein the concentrated liquid bioproduct has a concentration of viable cells of the strain ATCC® PTA-8805 of 10¹⁰-10¹² CFU/mL.

10. A bioproduct formulation according to claim 9, wherein the molasses culture medium comprises: water, 20 g/L sugar beet molasses, 2 g/L sucrose, 1 g/L yeast extract, 5 g/L ammonium sulfate ((NH₄)₂SO₄), 1 ml/L silicone antifoaming agent, 0.38 g/L dipotassium hydrogen phosphate (K₂HPO₄) and 11.25 g/L potassium dihydrogen phosphate (KH₂PO₄).

11. The method of using the concentrated liquid bioproduct formulation according to claim 8, wherein said bioproduct formulation is used to biologically control and decrease the incidence of Rhizoctonia solani, the causal agent of rhizoctoniasis in plants and black scurf in underground propagation clonal organs and underground roots.

12. The method of using the concentrated liquid bioproduct formulation according to claim 11, wherein said underground propagation clonal organs are potato tubers.

13. The method of using the concentrated liquid bioproduct formulation according to claim 11, wherein said underground roots are carrots or beets.
